(19) United States
(12) Plant Patent Application Publication
(10) Pub. No.: US 2004/0163152 P1
(43) Pub. Date: Aug. 19, 2004

(54) Aglaonema plant named 'Diamond Bay'

(76) Inventor: Richard J. Henny, Apopka, FL (US)

Correspondence Address: Thomas D. Stadsklev, Florida Foundation Seed Producers, Inc., P.O. Box 309, Greenwood, FL, 32443, US

(21) Appl. No.: 10/365,785

(22) Filed: Feb. 14, 2003

(51) Int. Cl. A01H 5/00

(52) U.S. Cl. PLT/376

(57) ABSTRACT

A new and distinct cultivar of Aglaonema named ‘Diamond Bay’, characterized by its large bicolored leaves with dark green borders and a solid gray central area, fairly compact growth habit, moderate branching, and with stems and petioles each consisting of two different colors.

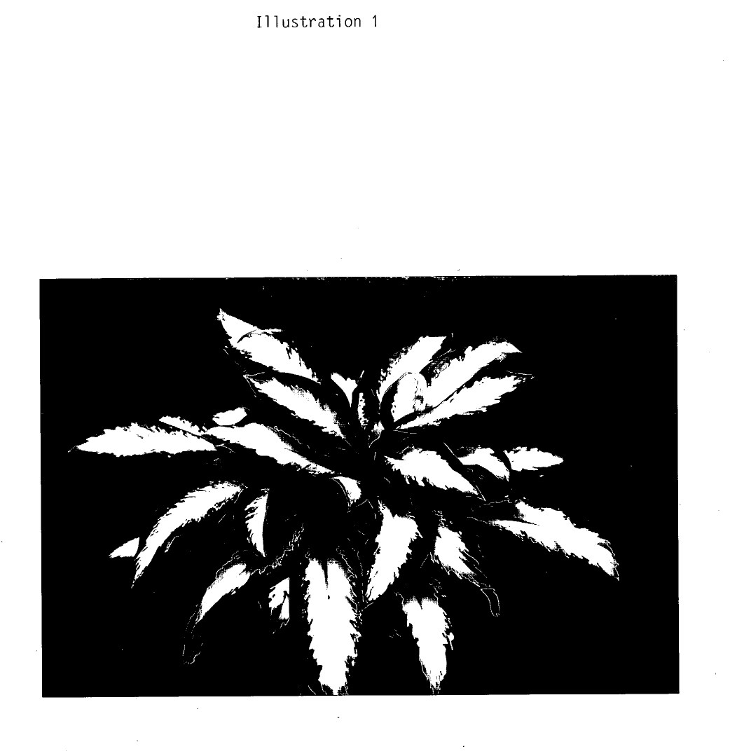

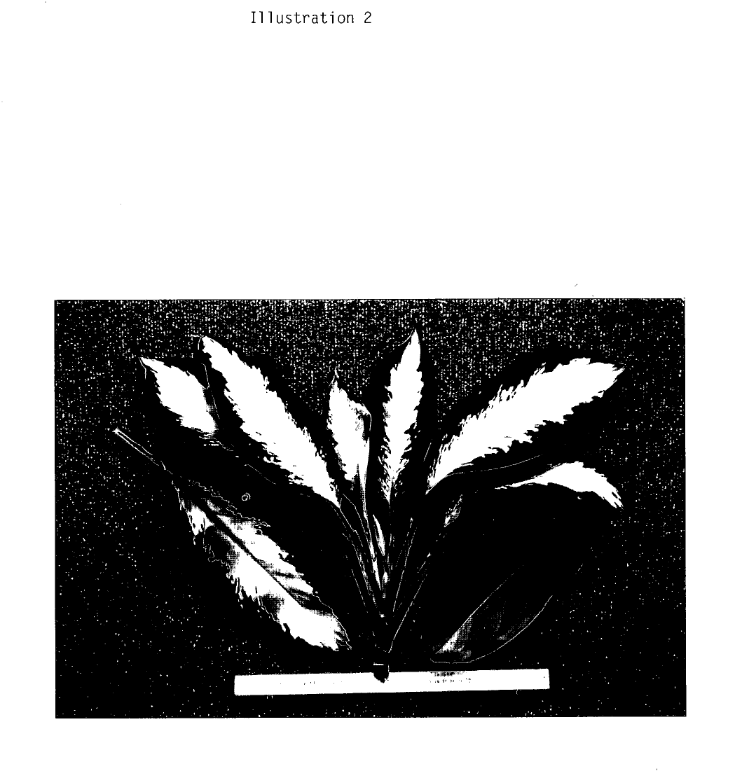

1. The upper surfaces of mature leaves are dark green with a distinct gray center area surrounding the leaf midrib that is half as wide as the leaf.

2. Plants are upright and somewhat outwardly arching in plant habit.

3. Plants are symmetrical and relatively compact and are suitable for 15 to 30 cm containers.

4. Plants are moderate in branching producing 2-4 basal shoots when grown from stem cuttings in controlled tests.

5. The stems and petioles each display two distinct colors.

### DESCRIPTION

The present invention relates to a new and distinct cultivar of Aglaonema plant, botanically known as Aglaonema hybrid, and hereinafter referred by the cultivar name ‘Diamond Bay’.

The new Aglaonema is a product of an extensive University of Florida ornamental tropical foliage plant breeding program conducted by the inventor, Dr. Richard J. Henny in Apopka, Fla. The objective of the breeding program is to develop new Aglaonemas with good growth habits and rate and novel appearance.

‘Diamond Bay’ was discovered and selected by the inventor as a sport from a large population of tissue-culture propagated Aglaonema ‘Silver Bay’ (a previously developed University of Florida hybrid cultivar) growing in a greenhouse in Apopka, Fla. Compared to plants of the original parent cultivar ‘Silver Bay’, leaves of mature plants of the new Aglaonema are longer and wider. Leaves of plants of the cultivar ‘Diamond Bay’ lack distinct gray bands along the leaf veins extending out beyond the central gray pattern. In addition, the stems and petioles of Diamond Bay display four distinct colors. Asexual propagation of the new cultivar by division, stem cuttings and tissue culture at Apopka, Fla. since 1999 has shown that the unique features of this new Aglaonema plant are stable and reproduced true to type in successive generations.

The new Aglaonema has not been observed under all possible environmental conditions. The phenotype may vary somewhat with variations in environment such as temperature, light intensity, fertilizer rate, and/or irrigation amount and frequency without, however, any variance in genotype. Plants were grown under greenhouse conditions with day temperatures ranging from 21 to 38 degrees C. and night temperatures ranging from 7 to 21 degrees C. The greenhouse shade provided approximately a 70 to 80 percent decrease in ambient light level. In the following description, color references are made to The Royal Horticultural Society Colour Chart (1995).

The following traits have been repeatedly observed and are determined to be the unique characteristics of ‘Diamond Bay’. These characteristics in combination distinguish ‘Diamond Bay’ as a new and distinct cultivar from other similar Aglaonema cultivars such as ‘Silver Moon’, ‘Rhapsody in Green’ and ‘Deborah’.

1. Plants of ‘Diamond Bay’ are relatively upright and somewhat outwardly arching in plant habit.

2. Plants of ‘Diamond Bay’ are fairly symmetrical and relatively compact and are suitable for 15 to 25-cm containers.

3. Plants of ‘Diamond Bay’ are moderate in branching, averaging 2-4 basal shoots when grown from stem cuttings in controlled tests.

4. The upper surfaces of mature leaves of ‘Diamond Bay’ are glossy silver green with dark green margins.

5. Both stems and petioles of ‘Diamond Bay’ display two distinct colors.

Diamond Bay differs from Aglaonema ‘Silver Moon’ (U.S. Plant Pat. No. 12,973) in the following ways:

1. ‘Diamond Bay’ is less compact than ‘Silver Moon’.

2. ‘Diamond Bay’ branches less than ‘Silver Moon’.

3. ‘Diamond Bay’ has two distinct colors on both the stem and petioles while ‘Silver Moon’ only has one on each.

‘Diamond Bay’ differs from Aglaonema ‘Deborah’ (U.S. Plant Pat. No. 9,975) in the following ways:

1. ‘Diamond Bay’ is more compact with shorter internodes and petioles than ‘Deborah’.

2. ‘Diamond Bay’ stems have no white coloration whereas ‘Deborah’ has white stems and petioles.

3. ‘Diamond Bay’ leaves have only two colors whereas ‘Deborah’ leaves are mottled and have a white midrib.

‘Diamond Bay’ differs from Aglaonema ‘Rhapsody in Green’ (U.S. Plant Pat. No. 8,975) in the following ways:

1. ‘Diamond Bay’ is more compact than ‘Rhapsody in Green’.

2. ‘Diamond Bay’ leaves are much wider than ‘Rhapsody in Green’.

3. ‘Diamond Bay’ stems and petioles each have two distinct colors whereas ‘Rhapsody in Green’ has only one.

### BRIEF DESCRIPTION OF THE DRAWINGS

The two color photographic drawings were taken from a typical plant of Aglaonema ‘Diamond Bay’ grown in a 20 cm diameter pot (3.9 liter volume), approximately 1 year after planting a 12-week-old rooted stem cutting with 5 leaves and grown under appropriate growing conditions. Colors are as accurate as possible with color illustrations of this type.

1. The first drawing depicts a top perspective view of a plant of ‘Diamond Bay’.

2. The second drawing depicts a mature stem and detached leaves to show how the upper leaf surface variegation is not visible on the lower leaf surface.

The following observations, measurements and comparisons describe plants grown in Apopka, Fla., in a shaded greenhouse and in conditions which closely approximate those used in horticultural practice. Plants were grown under day temperatures ranging from 21 to 38 degrees C. and night temperatures ranging from 7 to 21 degrees C. The greenhouse shade provided approximately a 70 to 80 percent decrease in ambient light level. In the following description, color references are made to The Royal Horticultural Society Colour Chart.

Origin: Sport of Aglaonema ‘Silver Bay’ (not patented).

Classification: Aglaonema cultivar ‘DiamondBay’.

Propagation: Asexual propagation either by stem cuttings, tissue culture or division.

Plant: In a 20 cm pot for a plant grown from a 12 week-old-cutting under appropriate growing conditions for 9 months, ‘Diamond Bay’ has an average canopy width of 55 cm and a canopy height of 33 cm. Mature leaves average 30.0 cm in length and 11.5 cm in width. Plants average 2-3 basal shoots.

Stem:

Growth pattern.—The stem is erect in growth and is 2.0 to 2.5 cm in diameter at five (5) cm above the soil surface. Internode distance is approximately 1.4 to 1.5 cm at five (5) cm above the soil.

Color.—The stem consists of two different colored areas that run together. The darker areas that are exposed to diffuse light are yellow-green (RHS 146B) and are roughly triangular in shape. These coalesce with areas of the stem that are a lighter shade of yellow-green (RHS 152B/C). The lighter areas are normally hidden by the attached clasping petiole.

Petiole: The following information is based on the 4^{th} expanded leaf from the apex of a one-year-old plant.

Dimensions.—Overall petiole length ranges from 14.0 to 22.0 cm. The petiole has expanded fleshy edges extending from the midrib and referred to as wings. The wings are approximately 2.0 to 2.4 cm, wide one-half the distance from the petiole base to the wing apex. The petiole wings average 10.0 to 16.0 cm in length and extend from the petiole base to within 3.0 to 9.0 cm of the leaf blade. The wings roll inward and are within 1.0 to 3.0 mm of the edges touching when not in contact with the stem. The angle between the stem and petiole is approximately 30 degrees from the stem axis for newer leaves. As leaves mature the angle becomes 40 to 50 degrees. The petiole and the leaf midrib form a near straight line on newly unfolding leaves. With maturity the leaf blades bend down to form an angle of 45 to 60 degrees between the petiole and the leaf underside. In some cases the oldest lower leaves may bend far enough to form an angle of 90 degrees.

Color.—The petiole wings consist of two colors including green (RHS 143C) on the outside edges blending to a lighter yellow-green (RHS 146B) in the center. The center petiole color extends up the leaf midrib on the leaf underside.

Leaf:

Growth pattern.—‘Diamond Bay’ leaves are elliptic and asymmetric in shape. Leaf bases are obtuse, tips are acuminate, and the margins are entire. There is some undulation in the leaves.

Dimensions.—Mature leaves may reach a length of 35 cm and a width of 16 cm although average leaves for mature plants grown in 20 cm pots are 30 to 32 cm in length and 11 to 13 cm in width (length/width ratio of 2.4 to 2.6).

Midrib.—The midrib is prominent and slightly recessed on the upper leaf surface and is the same color as the upper leaf blade surrounding it.

Color.—Mature leaves are bicolored. The two contrasting color zones consist of the central leaf zone that is gray-green (RHS 191C/D) in an area, centered around the leaf midrib, that covers approximately one half of the upper leaf surface. The patterned edges are irregular but clearly defined. Outer edges of the upper leaf surface are dark green (RHS 147A) and this color extends inward approximately 2 to 3 cm where it meets with the central gray zone. Occasionally a few small (1 cm or less in any direction) irregular darker islands (RHS 147A/B) appear within the lighter gray central leaf area. Conversely, a few lighter colored (RHS 191C/D) islands appear in the darker green outer leaf border. A uniform green (RHS 147B) covers the leaf blade and leaf midrib on the underside.

Inflorescence: Although the Aglaonema inflorescence has no commercial value the following description applies. Spathes and spadices are held on arching peduncles and there may be 3 to 6 inflorescences produced at each flowering node. Spathes range in length from 7.2 to 9.8 cm in length and 1.0 to 2.0 cm in width. With the spathe removed, the female portion of the inflorescence ranges from 1.0 to 2.0 cm in length and the male portion may be 4.0 to 5.0 cm in length. The number of female flowers per inflorescence ranges from 15 to 22. Inflorescences may appear sporadically once plants reach 9 months of age. The outer spathe color is yellow-green (RHS 145A). Again, with the spathe removed, the male portion of the spadix is grayed-orange (RHS 163C) in new inflorescences and darkens to grayed-orange (RMS 165B) with age. The female flower stigma is yellow (RHS 13B) while the female ovary is yellow-white (RHS 158B). Inflorescences are open for 2 to 3 days after which the spathe closes. Subsequently inflorescences wither and die within 4 to 6 weeks of opening.

Seed: No seed has been observed on ‘Diamond Bay’.

Roots: Roots are typical for Aglaonema being thick white roots with fine laterals. The root system is very vigorous.

Disease/insect resistance: In trial tests under commercial production conditions plants of Aglaonema ‘Diamond Bay’ have not shown any unusual susceptibility to diseases or insects common to Aglaonema.

1. A new and distinct cultivar of Aglaonema named ‘Diamond Bay’, characterized by its large bicolored leaves with dark green borders and a solid gray central area, fairly compact growth habit, moderate branching, and with stems and petioles each consisting of two different colors.
